(19) United States
(12) Patent Application Publication
(10) Pub. No.: US 2002/0052692 A1
(43) Pub. Date: May 2, 2002

(54) COMPUTER SYSTEMS AND METHODS FOR HIERARCHICAL CLUSTER ANALYSIS OF LARGE SETS OF BIOLOGICAL DATA INCLUDING HIGHLY DENSE GENE ARRAY DATA

(76) Inventor: EOIN D. FAHY, SAN DIEGO, CA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/397,380

(22) Filed: Sep. 15, 1999

(51) Int. Cl. G01N 33/48; G01N 15/06; G06F 7/00

(52) U.S. Cl. 702/19; 422/68.1; 707/100

(57) ABSTRACT

A system and corresponding method analyzes biological data for sets of test subjects such as gene arrays of group test subjects into clusters and order the clusters into a hierarchy based on similarities and differences of biological data corresponding to the test subjects. A combination of nonhierarchical clustering and hierarchical clustering methods is used to efficiently and effectively perform hierarchical clustering of such biological data as highly dense gene arrays containing many thousand test subjects such as genes. First the test subjects are nonhierarchically clustered according to similarities and differences of their biological data as determined by distance techniques. Representative values, such as mean values, of the biological data are determined for each nonhierarchical cluster of test subjects. These representative values are then used to hierarchically cluster the nonhierarchical clusters. Biological data for each test subject is displayed in a row of a table. The rows of the table are arranged by the nonhierarchical clustering and further by the hierarchical clustering. Each value of the biological data is color coded according to its value to display patterns in the hierarchically clustered biological data.

### TECHNICAL FIELD

The present invention relates to computer systems and methods for analyzing sets of data, and more particularly, to computer systems and methods for cluster analysis of large sets of biological data including gene array data.

### BACKGROUND OF THE INVENTION

Hierarchical cluster analysis has been used in biology to assist in discovering correlations and patterns in data. It can be useful, for instance, when a set of measurements is performed on each test subject of a group resulting in a test matrix. Typically, each row of a test matrix will be assigned to an individual test subject of the group of test subjects and each column of a test matrix will be assigned to an individual measurement of the set of measurements. The intersection of a row and a column of the test matrix will then contain a value for a particular measurement of the set of measurements performed on a particular test subject of the group of test subjects.

For instance, a test matrix could contain a set of measurements for each animal of a group of animals. Individual rows of the test matrix would be assigned to individual test subject animals such as elephants, dogs, and hamsters. Individual columns of the test matrix would be assigned to individual measurements of the test subjects, such as size, weight, running speed, and hours of sleep. For instance, a particular intersection of the row assigned to the elephant test subject with the column assigned the weight measurement would contain a value for the weight of the elephant. This weight value may be the weight of a particular elephant, a statistical value representing weight of a particular group of elephants or other value related to weight of elephant test subjects such as an arithmetic or logarithmic ratio of average weight of elephants with a particular disease compared to average weight of a control group of healthy elephants.

Performing hierarchical cluster analysis on a test matrix can be helpful in organizing in a hierarchical fashion a group of test subjects based upon measurement values associated with the test subjects. This hierarchical ordering of the group of test subjects can be visually represented by a dendrogram. A representative example of a dendrogram, shown in FIG. 1, contains a total of eight test subjects. Based on measurements of height, weight, top speed, and hours of sleep of the test subjects, a hierarchical cluster analysis may order the rhinoceros and elephant together into a first cluster and the horse alone in a second cluster. The hierarchical cluster analysis could then order the first and second clusters into a third cluster. The hierarchical cluster analysis may also first order a dog and a cat into a fourth cluster, a rat and a hamster into a fifth cluster and a mouse alone into a sixth cluster. The hierarchical cluster analysis could then group the fourth, fifth, and sixth clusters into a seventh cluster resulting in two main clusters: the third and seventh clusters as shown in the dendrogram of FIG. 1.

Hierarchical cluster analysis has also been computer automated. Satisfactory results occur with computer automated hierarchical clustering when the number of test subjects is relatively small and also the number of measurements performed is relatively few. Computer automated hierarchical clustering has not performed well, however, for large numbers of test subjects because of unrealistic demands on system capacity to perform the cluster analysis. Also, as the number of test subjects increases, representation of a resultant hierarchical clustering with a dendrogram becomes less visually effective since the number of branches and sub-groups grows tremendously to the point that the human eye can no longer perceive patterns and correlations in the measurements.

This breakdown in effectiveness of conventional computer automated hierarchical clustering is very evident in the growing field of genetic testing. Improvements in genetic testing facilities are occurring at a rapid pace, such that new test matrices can contain genetic testing data from 5,000 to 20,000 test subjects or more usually with numerous measurements for each test subject. This size of test matrix requires extraordinarily vast computer resources (e.g., memory, storage, processing speed) for conventional computer automated hierarchical clustering, where such resources exceed the capabilities of those typically used by genetic researchers, scientists and others. Yet, computer automated hierarchical clustering has great potential to assist such investigators if they could reasonably perform computer automated hierarchical clustering using typical computer resources that are commonly available to them.

A further problem regarding conventional computer automated hierarchical clustering for the large matrices of genetic data relates to the manner in which hierarchical clustering is visually represented by dendrograms. Even for a hundred test subjects, an associated dendrogram appears very complex to the human eye and only gross patterns may be evident. With modern genetic test data having test matrices with tens of thousands of test subjects, a dendrogram representation of the data no longer is effective for a human visually to perceive correlations and patterns buried in the dendrogram representation of the data. Also, dendrograms in general fail to give enough detail to show why certain test subjects are clustered together.

Without a way to perform computer automated hierarchical clustering on computer resources that are typically available to researchers, scientists, and others, and without a way to visually represent results of hierarchical clustering meaningfully to the human eye, discoveries and breakthroughs in understanding of genetics with respect to such areas as human disease and suffering will go unnoticed and unappreciated by those in positions to beneficially teach and influence others in society. For example, hierarchical cluster analysis of gene expression in patients suffering from a disorder that has multiple potential genetic components may be used by a physician in the course of designing a treatment regimen that is best suited for particular subpopulations of such patients, or by a research scientist to discern which sets of genes, and associated biochemical pathways, might be useful for discovering therapeutic compositions for such disorders.

### SUMMARY OF THE INVENTION

The present invention overcomes the limitations of the prior art and provides additional benefits. One aspect of the invention is directed to a system for analyzing information based on measurements of a plurality of measurement types. A measurement of each measurement type is performed on each of a plurality of biological test subjects. An input component is configured to receive a data file of a test matrix containing sets of measurement values. Each set of measurement values contains a measurement of each measurement type for one of the plurality of biological test subjects.

Other aspects of the system include a preconditioning component configured to assign each of the sets of measurement values to a plurality of nonhierarchical clusters. At least one of the nonhierarchical clusters has more than one set of measurement values assigned. Additional aspects of the system include a reduction component configured to generate a data file of a reduced test matrix from the data file of the test matrix. The reduced test matrix contains one set of representative values associated with each nonhierarchical cluster. Each set of representative values is based on the sets of measurement values assigned to the nonhierarchical cluster associated with each set of representative values. Further aspects include a hierarchical clustering component configured to order the sets of representative values into hierarchical clusters.

Additional aspects of the invention include an analysis system comprising receiver and clustering components. A data conditioning system comprises an input component and a conversion component. A computer-readable medium stores instructions comprising inputting a data file, assigning biological sample data, generating representative values, and ordering nonhierarchical clusters. A system for displaying hierarchical clusters comprises a color monitor, a computer system, and a software program configured to instruct the computer system to display nonhierarchically clustered values. Additional aspects include a data structure containing information generated from biological samples or methods for generating information based on biological samples or displaying biological data records or representative records.

### BRIEF DESCRIPTION OF THE DRAWINGS

FIG. 1 is a dendrogram illustrating prior art visual representations of hierarchical cluster analysis.

FIG. 2 is a block diagram of a computing system suitable for applying aspects of the invention to analyze a cluster biological data.

FIG. 3 is a flowchart depicting a method of hierarchically clustering test matrices according to the present intervention.

FIG. 4 is a display screen showing a portion of the spreadsheet table containing hierarchically clustered gene array data with values color-coded according to value ranges.

FIG. 5 is a portion of a dendrogram corresponding to the portion of the spreadsheet table shown in FIG. 4.

FIG. 6 is a display screen showing a portion of the spreadsheet table containing hierarchically clustered values based on nonhierarchically clustered gene array data.

FIG. 7 is a display screen showing a dialog box for entering queries of a database containing hierarchically clustered gene array data.

FIGS. 8 and 9 are display screens showing a portion of results of a database queries having a hierarchically clustered gene array data color-coded according to value ranges.

### DETAILED DESCRIPTION OF THE INVENTION

A data analysis system, and in particular, a computer system and method for efficiently ordering large amounts of biological data into hierarchical clusters and effectively displaying the resultant order is described in detail herein. In the following description, numerous specific details are provided, such as specific input data, preconditioning methods, and nonhierarchical and hierarchical clustering methods, display screen layouts, etc., to provide a thorough understanding of embodiments of the invention. One skilled in the relevant art, however, will recognize that the invention can be practiced without one or more of these specific details, or with other input data, preconditioning methods, or nonhierarchical or hierarchical clustering methods, display screens, etc. In other instances, well-known structures or operations are not shown, or not described in detail, to avoid obscuring aspects of the invention.

Genetics researchers are concerned with the molecular tools and instructions used by cells of human and animal bodies to make proteins found in these cells. Proteins are a class of molecules that include many important biological structural and functional components, for example, tissue material, enzymes, and hormones. To make a protein, cells possess complex and specialized molecular machinery that assembles linear polymers of different amino acids that are strung together in assorted lengths and orders to generate hundreds of thousands of proteins. Not all cells produce the same types of proteins or the same amounts. For instance, stomach cells do not normally synthesize hair proteins, whereas other cells (e.g., specialized cells of the skin and scalp) normally do produce hair proteins. Depending on the condition of the individual human or animal, certain cells may produce more or less protein of different types.

There are 20 naturally occurring amino acids that are used by the cells to make the various proteins. Remarkably, each cell normally has a full set of genetic instructions to assemble the amino acids for all the proteins produced by the body. For instance, stomach cells contain the instructions for making hair proteins even though stomach cells would not normally synthesize hair proteins.

This set of instructions is found in large molecules known as deoxyribonucleic acid (DNA), which is a major component of chromosomes found in cells. A DNA molecule contains regions called genes, many of which contain instructions for making a particular protein. Part of the gene region also provides a sensor for molecular signals that determine whether the gene is expressed above baseline levels (i.e., the gene is turned “on”) or expressed only at baseline levels or less (i.e., the gene is turned “off”). For instance, in a stomach cell, a gene involved in making hair protein would normally be turned “off” so that no hair protein would be made by the stomach cell whereas in appropriately specialized cells of another region (e.g., skin, scalp) genes for making hair protein would be turned “on.” Typically, certain genes in a cell are always turned “off” whereas other genes in the cell are sometimes “on” and at other times “off.”

Genetic researchers are concerned with what genes are turned “on” and “off” for different types of cells and under different conditions. Since a single isolated cell is relatively difficult to study, typically, groups of cells are studied. Values are then used to represent the portions of cells in a group that are in the “on” and “off” state. For instance, if not all cells of a group of cells have a certain gene for making a particular protein turned “on” in a region, resulting values may be lower or higher than if all the genes were turned “on.”

Furthermore, some cells may have certain genes modified so that either the genes instruct their cells to produce proteins of an incorrect type or these modified genes may be simply turned “off” even though these genes in a healthy cell of the same type would normally be turned “on.” These genes could have been modified by some accidental exposure to radiation, toxic chemicals or other harm. These modified genes could also have been inherited from a parent of the human or animal with the modified genes.

Genetic researchers have devised ways of producing a “gene array,” in which probes for nucleotide sequences that are specific for particular genes are arranged, based on a predetermined design, onto a supporting medium such as, for example, specially produced paper, nylon, nitrocellulose, silicon, glass, cellulose acetate or any other suitable material known in the art. Nucleic acids (e.g., DNA or RNA) can be prepared from cells or tissue samples derived from a subject in order to provide test samples that are tested (probed) by being contacted with the gene array. It is also possible to immobilize the nucleic acids of the test sample on a suitable supporting medium which is then contacted with the probes; in this situation, the gene array is a mixture, which may be a solution, of probes for specific nucleotide sequences. In another version of the gene array, a physical array is not present per se but the amounts in the test samples of a set of nucleotide sequences that are specific for particular genes are determined by methods known in the art such as, e.g., Southern analysis, Northern analysis, quantitative polymerase chain reaction (PCR). In any event, each probe of the gene array selectively reacts with nucleic acids in the test sample that have a nucleotide sequence nucleotide sequence. Regardless of the nature of the reaction, the extent of which depends upon the degree to which the particular gene is turned “on” or “off,” it produces a signal that is detected and read by a device that generates a nucleic acid expression data file comprising the results (i.e., absolute or relative amounts of particular nucleic acids in samples). There are some 70,000-100,000 different genes in the DNA present within a typical mammalian cell. Currently, there are approximately 20,000 probes to test activity levels for particular genes. In the future, this number of probes is likely to increase.

The probe reactions in the gene arrays can be analyzed efficiently to produce highly dense gene array data having many thousands of values to indicate activity levels of many thousands of different genes. An activity level value for a particular gene type would be based upon how many genes of the particular gene type from a group of cells would be turned “on” to instruct the cell to make a particular type of protein.

The data file that the input component receives may additionally or alternatively be based upon a “protein array.” In a protein array, probes for specific polypeptides are arranged on a suitable supporting material, and a test sample derived from a subject and comprising proteins therefrom is contacted with the probes. Each probe of the protein array selectively reacts (hybridizes) with proteins in the test sample that have a specific amino acid sequence, and the extent of these reactions depends upon the amount of a particular protein present in the test sample. One type of probe suitable for such an array is an antibody, which may be a polyclonal, monospecific or monoclonal antibody. Alternatively, the protein test sample may be analyzed by techniques such as, e.g., two-dimensional gel electrophoresis or HPLC, in order to characterize some or all of the test proteins and their amino acid sequences. Regardless of what type of protein array is used, a particular protein results in a signal, the extent of which depends upon the degree to which the particular protein is present in the sample, and the production of that signal is detected and read by a device that generates a protein expression data file comprising the results (i.e., absolute or relative amounts of particular proteins in samples).

It is also possible to utilize data files that combine results from gene arrays, protein arrays, and other sources of information (such as, e.g., information from patient profiles regarding such parameters as the subject's weight, age, gender, diet, drug treatments, etc.) in the present invention. Thus, the present invention allows an artisan to generate hierarchical cluster analysis with regards to, for example, the expression of a particular set of genes, and the proteins encoded thereby, in all the subjects in a clinical drug study who are male, over 40 years of age, under 150 pounds in weight that smoke a defined number (or range) of cigarettes per day. In this example, such analyses will allow artisans to identify trends and patterns that will assist them in deciding which treatment regimens are most appropriate for which patients.

These thousands of values are then assembled into a test matrix with the rows representing individual genes as the test subjects and the columns representing gene activity measurements associated with factors such as different conditions, regions, or environments of the genes including cell type, tissue type, disease type, structural family type, functional family type, and time points or particular input parameters of an individual experiment. For instance, a row of the test matrix could be the gene that instructs the cell to make a particular enzyme and a column could be genes from liver cells in a particular area of the liver. The intersection of this column and row would contain a value representing the amount of activity for genes involved with production of the particular enzyme found in cells of the particular area in the liver.

Researchers can further refine the gene activity values found in a test matrix by generating values based upon gene activity of individuals of a study group compared with gene activity of a control group. In comparing study groups with control groups a ratio is typically taken of the study values over the control values. This resulting ratio value can then be expressed in terms of logarithms so that that negative and positive numbers will reflect the ratios being less than or greater than one. Oftentimes, the individuals of the study group have some form of disease whereas the individuals of the control group do not have the form of disease.

The resulting test matrices from modern gene array methods of genetic research can be very large having thousands of rows of activity values for thousands of individual genes. Conventional computer automated hierarchical clustering methods is limited to approximately 1,000 rows of data using typical computer resources available to the researchers before serious performance issues arise causing unacceptable delays or failures in processing the enormous test matrices.

Aspects of the present invention overcomes the limitations of the prior art and is directed to systems and methods for hierarchical clustering of large test matrices having over approximately 1,000 rows or more of data with less demanding computer resource requirements than the prior art. Aspects of the present invention display results from its form of hierarchical clustering in systems and methods that are more adept at conveying information regarding hierarchical clustering of large test matrices than the conventional dendrograms of the prior art. While the depicted embodiment of the present invention is directed to gene arrays, other embodiments are directed to other forms of biological data such as expressed sequence tags (see, e.g., www.ncbi.nlm.nih.gov/dbEST) and full-length sequences related to DNA research (see, e.g., www.nchi.nlm.nih.gov/Entrez/nucleotide.html).

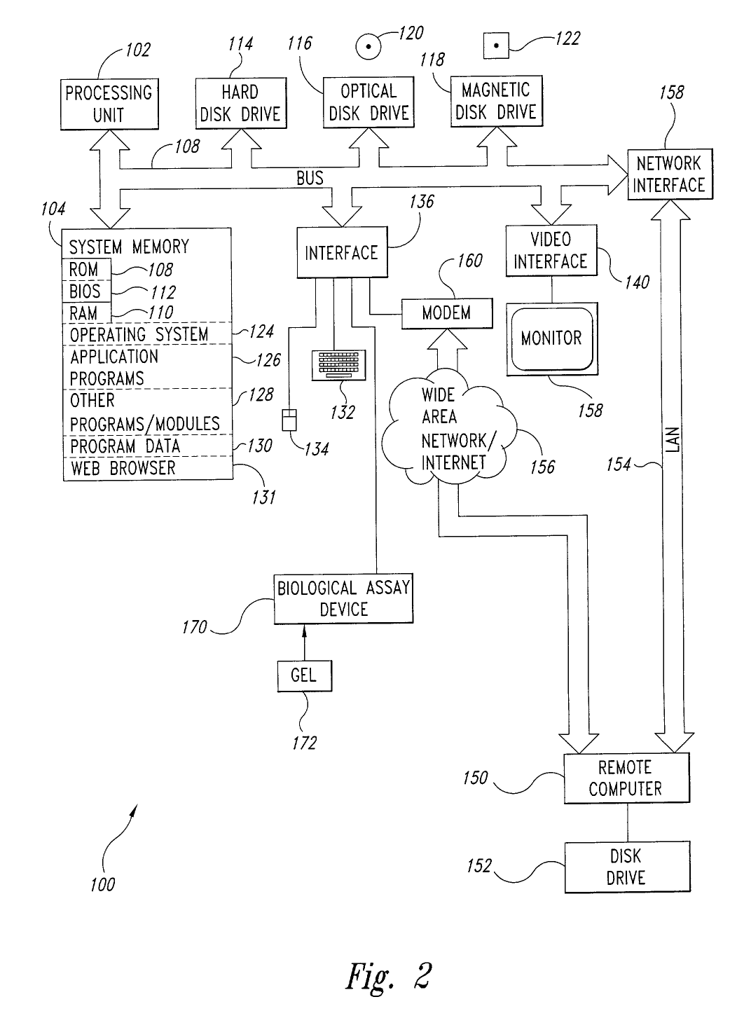

FIG. 2 and the following discussion provide a brief, general description of a suitable computing environment in which the invention can be implemented. Although not required, embodiments of the invention will be described in the general context of computer-executable instructions, such as program modules or macros being executed by a personal computer. Those skilled in the relevant art will appreciate that the invention can be practiced with other computer system configurations, including hand-held devices, multiprocessor systems, microprocessor based or programmable consumer electronics, networked PCs, mini computers, mainframe computers, and the like. The invention can be practiced in distributed computing environments where tasks or modules are performed by remote processing devices, which are linked through a communication network. In a distributed computing environment, program modules may be located in both local and remote memory storage devices.

Referring to FIG. 2, a conventional personal computer 100 includes a processing unit 102, a system memory 104 and a system bus 106 that couples various system components including the system memory to the processing unit. The processing unit 102 may be any logic processing unit, such as one or more central processing units (CPUs), digital signal processors (DSPs), application-specific integrated circuits (ASIC), etc. Unless described otherwise, the construction and operation of the various blocks shown in FIG. 2 are of conventional design. As a result, such blocks need not be described in further detail herein, as they will be understood by those skilled in the relevant art.

The system bus 106 can employ any known bus structures or architectures, including a memory bus with memory controller, a peripheral bus, and a local bus. The system memory 104 includes read-only memory (“ROM”) 108 and random access memory (“RAM”) 110. A basic input/output system (“BIOS”) 112, which can form part of the ROM 108, contains basic routines that help transfer information between elements within the personal computer 100, such as during start.

The personal computer 100 also includes a hard disk drive 114 for reading from and writing to a hard disk (not shown), and an optical disk drive 116 and a magnetic disk drive 118 for reading from and writing to removable optical disks 120 and magnetic disks 122, respectively. The optical disk 120 can be a CD-ROM, while the magnetic disk 122 can be a magnetic floppy disk. The hard disk drive 114, optical disk drive 116 and magnetic disk drive 118 communicate with the processing unit 102 via the bus 106. The hard disk drive 114, optical disk drive 116 and magnetic disk drive 118 may include interfaces or controllers (not shown) coupled between such drives and the bus 106, as is known by those skilled in the art. The drives 114, 116 and 118, and their associated computer-readable media, provide nonvolatile storage of computer-readable instructions, data structures, program modules and other data for the personal computer 100. Although the depicted personal computer 100 employs a hard disk, optical disk 120 and magnetic disk 122, those skilled in the relevant art will appreciate that other types of computer-readable media that can store data accessible by a computer may be employed, such as magnetic cassettes, flash memory cards, digital video disks (“DVD”), Bernoulli cartridges, RAMs, ROMs, smart cards, etc.

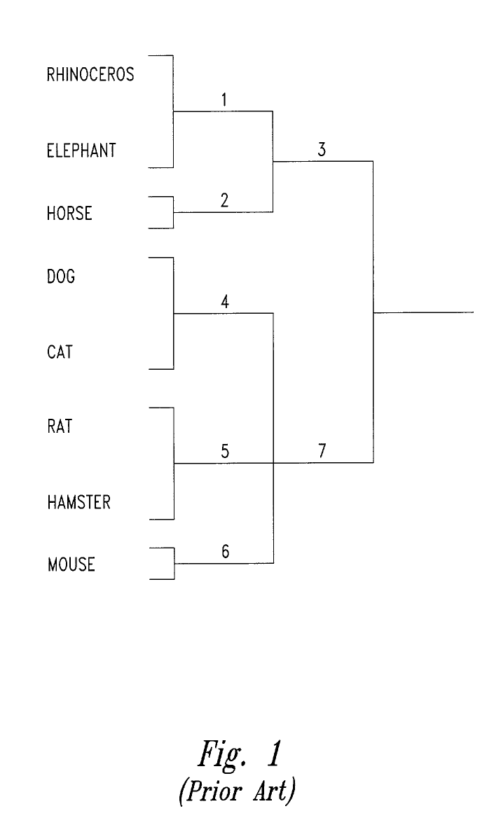

Program modules can be stored in the system memory 104, such as an operating system 124, one or more application programs 126, other programs or modules 128 and program data 130. The system memory 104 may also include a web browser 131 for permitting the personal computer 100 to access and exchange data with web sites in the World Wide Web of the Internet, as described below. The application programs 126 can include spreadsheet applications, such as Excel® by Microsoft Corp. While shown in FIG. 1 as being stored in the system memory 104, the operating system 124, application programs 126, other modules 128, program data 130 and web browser 138 can be stored on the hard disk of the hard disk drive 114, the optical disk 120 of the optical disk drive 116 and/or the magnetic disk 122 of the magnetic disk drive 118.

A user can enter commands and information into the personal computer 100 through input devices such as a keyboard 132 and a pointing device such as a mouse 134. Other input devices (not shown) can include a microphone, joystick, game pad, scanner, etc. These and other input devices are connected to the processing unit 102 through an interface 136 such as a serial port interface that couples to the bus 106, although other interfaces such as a parallel port, game port or universal serial bus (“USB”) can be used. A monitor 138 or other display device is coupled to the bus 106 via a video interface 140, such as a video adapter. The personal computer 100 can include other output devices, such as speakers, printers, etc.

The personal computer 100 can operate in a networked environment using logical connections to one or more remote computers, such as a remote computer 150. The remote computer 150 can be another personal computer, a server, a router, a network PC, a peer device or other common network node, and typically includes many or all of the elements described above for the personal computer 100. Typically, the remote computer 150 includes a memory storage device such as a disk drive 152 shown in FIG. 1. The remote computer 150 is logically connected to the personal computer 100 under any known method of permitting computers to communicate, such as through a local area network (“LAN”) 154 or a wide area network (“WAN”) or Internet 156. Such networking environments are well known in offices, enterprise-wide computer networks, Intranets, and the Internet.

When used in a LAN networking environment, the personal computer 100 is connected to the LAN 154 through an adapter or network interface 158 (coupled to the bus 106). When used in a WAN networking environment, the personal computer 100 often includes a modem 160 or other device for establishing communications over the WAN/Internet 156. The modem 160 is shown in FIG. 1 as coupled between the interface 136 and the WAN/Internet 156. In a networked environment, program modules, application programs, or data, or portions thereof, can be stored in the remote computer 150, such as in the disk drive 152. Those skilled in the relevant art will readily recognize that the network connections shown in FIG. 1 are only some examples of establishing communication links between computers, and other links may be used, including wireless links.

A biological assay device 170, such as one that produces highly dense gene array data, can be coupled to the personal computer 100, such as through the interface 136. Those skilled in the relevant art will readily know how to generate highly dense gene arrays with corresponding test matrices having from 1,000 to 20,000 or more test subject rows, each row having measurement values related to activity of a gene as a test subject. Although the depicted embodiment uses test subject rows, alternative embodiments use columns for test subjects and rows for measurements.

Test matrices can be supplied having raw data for a particular study or control group to the personal computer 100 for further processing either alone or with other test matrices having gene activity values representing gene arrays from both study and control groups. Alternatively, test matrices having pre-processed gene activity values representing gene arrays from both study and control groups can be supplied to the personal computer.

In the depicted embodiment, the personal computer 100 performs hierarchical analysis on test matrices containing gene activity values representing gene arrays for both study and control groups. Alternative embodiments of hierarchical analysis order test matrices having gene activity values representing single gene arrays or various other combinations of gene arrays.

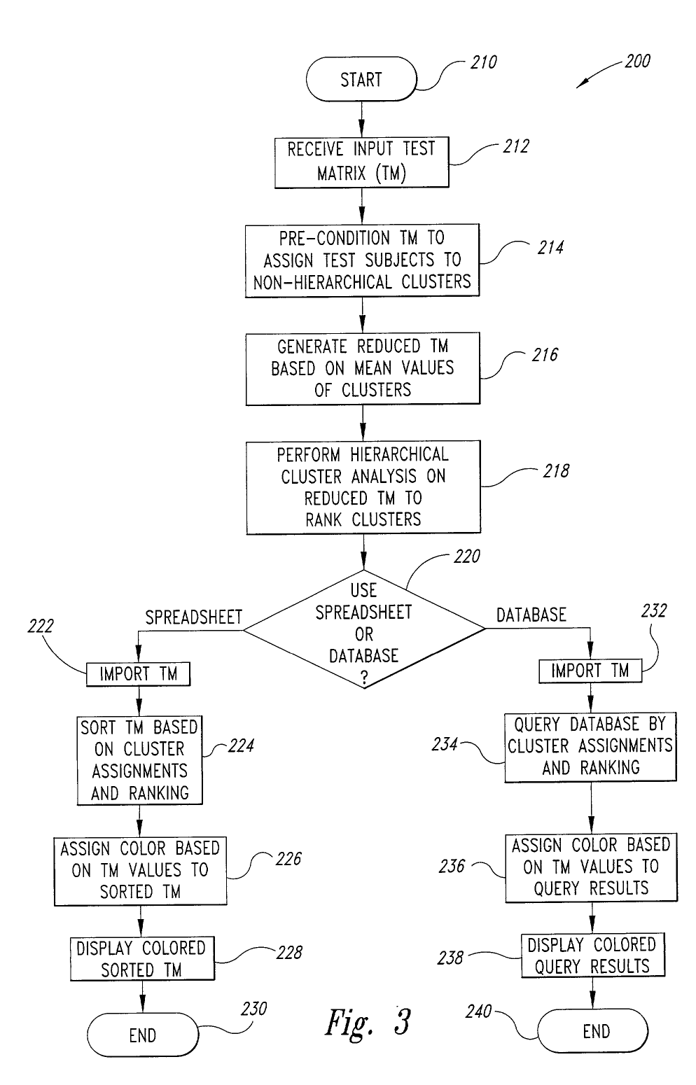

Once a test matrix is chosen for hierarchical clustering, the depicted embodiment uses the procedure 200 as shown in FIG. 3 to hierarchical cluster the test matrix. The hierarchical clustering procedure 200 begins at step 210 and proceeds to step 212 where a test matrix is inputted into or received by the personal computer 100 for analysis. In the depicted embodiment, test matrices are inputted in ASCII text file format. Alternative embodiments use other file formats for test matrix input.

In step 214, the procedure 200 preconditions the test matrix by assigning the test subject rows of the test matrix to nonhierarchical clusters. Nonhierarchical clustering forms the test subject rows into separate groups or clusters, but does not order these clusters in a hierarchy. During nonhierarchical clustering, the nonhierarchical clusters remain independent from one another and are not associated with each other or clustered together to form larger clusters. Under the depicted embodiment, the procedure 200 uses K-means clustering, which is a conventional nonhierarchical clustering method. In particular, for the depicted embodiment, the procedure 200 uses a known Linux freeware statistical software package entitled “R” to perform the K-means clustering under step 214. Further information on R is found on the Internet at http://lib.stat.cmu.edu/R/.

The K-means clustering procedure uses a preselected number, K, as the number of clusters to form. The K-means clustering procedure then produces exactly K different clusters of the test sample rows of greatest possible distinction. The K-means procedure tests a number of different groupings of the test subject rows into nonhierarchical clusters to search for a set of clusters that maximizes the similarity of all the test sample rows assigned to the same cluster. At the same time, the K-means procedure maximizes the statistical distance or differences between individual clusters. The depicted embodiment uses Euclidean distances between the various gene activity values of the test subject rows of a test matrix to determine distances between the test sample rows. The K-means clustering procedure under the above embodiment then uses a value for K of 200 for test matrices of approximately 20,000 test subject rows. When using the K-means procedure under the R statistical package, the results are then formatted using the Perl scripting language for input into the next step of the overall hierarchical clustering procedure. Perl is a conventional UNIX utility well known in the art. Pern is an interpreted language directed to system management tasks and other aspects directed to scanning computer files, extracting information from those files, and printing and formatting the files based on that information.

Although the above embodiment uses K-means clustering with Euclidean distances for step 214 to precondition the test subject rows, alternative embodiments use other nonhierarchical clustering methods and/or other distance measurements. For instance, instead of Euclidean distance determinations, other distance determination methods can be used such as squared Euclidean, Cosine, Pearson correlation, Chebychev, Block, and Minkowski, which are known.

Other aspects of K-means nonhierarchical clustering include various methods of choosing cluster seeds to initiate cluster formation. These methods include sequential threshold, parallel threshold, and optimization. Under the sequential threshold method, the procedure 200 selects a first one cluster seed, being a test subject row, to include all test subject rows within a pre-specified distance. When the procedure 200 includes all test subject rows within the distance, the procedure 200 selects a second cluster seed to include all test subject rows within the pre-specified distance. The procedure 200 repeats the selection process until all test subject rows have been included in a cluster. Under the parallel threshold method, the procedure 200 selects several cluster seeds simultaneously and assigns test subject rows within a threshold distance to the nearest seed. Under the optimization method, the procedure 200 allows reassignments of test subject rows in an iterative fashion.

K-means nonhierarchical clustering is one method using an unsupervised classification scheme. Another nonhierarchical clustering method using unsupervised classification is Isodata also known in the art and used by alternative embodiments. Isodata iteratively clusters based on mean values of current clusters and minimum distance techniques. The Isodata method stops when a number of test subjects in each cluster changes by less than a threshold or when a maximum number of iterations is reached.

Once nonhierarchical clustering is performed, the procedure 200 gives nonhierarchical cluster assignments to each test subject row of the test matrix. As a result of these assignments, the test matrix contains an additional column wherein each test subject row contains an additional entry indicating its nonhierarchical cluster assignment. Under the above embodiment, the personal computer 100 uses a Perl script to perform these manipulations on the data file containing the test matrix.

For test subject rows that share the same nonhierarchical cluster assignment, the personal computer 100 determines mean values for each measurement to generate a row of a reduced test matrix in step 216 of FIG. 3. The resultant reduced test matrix thus contains reduced matrix rows that contain mean values for each nonhierarchical cluster of test matrix rows.

In step 218, the personal computer 100 performs hierarchical cluster analysis on the reduced test matrix to order the nonhierarchical cluster assignments. The hierarchical cluster analysis uses the mean values of the reduced matrix rows from the reduced test matrix as the basis for the hierarchical clustering. For instance, the above embodiment uses “200” for the value of K in nonhierarchical clustering. Consequently, the hierarchical cluster analysis will cluster “200” reduced matrix rows under step 218. In hierarchical cluster analysis, the personal computer 100 decides at each iteration how the clusters are to be merged. For N reduced matrix rows, there are N−1 merges and 2^{N-1} possible orderings for the branches in a cluster tree, or dendrogram.

The above embodiment uses HClust based on Fortran code written by F. Murtagh and found in Statlib from the R statistical package. HClust provides a set of procedures to perform hierarchical clustering and to manipulate the resulting dendrogram. Many different cluster methods are offered by HClust to amalgamate the clusters. Here HClust determines when cluster pairs are sufficiently similar to be linked together to form a single cluster based upon a particular formulation of the values of the clusters involved, which is referred to as a “distance” between the clusters. Under an agglomerative approach, initially each reduced test matrix row is assigned its own cluster. Then HClust proceeds to iteratively join pairs of reduced test matrix rows into clusters first and then join pairs of clusters into larger clusters until there is just a single cluster. At each stage, the personal computer 100 recomputes distances between clusters, for instance, by the Lance-Williams dissimilarity update formula according to the particular clustering method being used.

In general, a hierarchical cluster method determines distances between clusters of cluster pairs to amalgamate clusters. These distances reflect the dissimilarities between the clusters of a cluster pair. Distances are determined with respect to the cluster objects. In the case of the above embodiment of the present invention, these objects are the reduced matrix rows. Distances are determined with respect to single dimensions or multiple dimensions. In terms of hierarchical cluster analysis, the various categories of values of a reduced matrix row are viewed as dimensions. That is, dimensions of hierarchical analysis refer to the types of measurements of, for instance, gene activity. As previously stated, these types of measurements are based on, for example, different test conditions, regions, or environments of the genes including cell type, tissue type, disease type, structural family type, functional family type, and time points or particular input parameters of an individual experiment.

A common way of determining distances is by the Euclidean distance method. Here distance is computed as the actual geometric distance between objects in N-dimensional space where the objects are reduced matrix rows. Alternative embodiments employ other ways of determining distances between clusters, including Squared Euclidean distance, City-block or Manhattan distance, Chebychev distance, Power distance, and Percent disagreement methods known in the art.

HClust offers many agglomeration methods including average, Ward, single, complete, Mcquitty, median, or centroid having different ways of defining what cluster objects to use in determining distance between the cluster pair. The average method is the default choice of HClust and is used in the above embodiment, although alternative embodiments employ other methods as noted below.

Two forms of the average agglomeration method are the unweighted pair-group average, and the weighted pair-group average. With unweighted pair-group average the distance between a cluster pair is calculated as the average distance between all pairs of objects in the cluster pair. This method is typically suitable when either the objects of the clusters form naturally distinct groups or when they form elongated chains. This method is also referred to as the unweighted pair-group method using arithmetic averages. The weighted pair-group average method differs from the unweighted pair-group average method in that the size of respective clusters determines a weight to be applied to the respective clusters. Cluster size is based on the number of objects contained in a cluster.

The Ward's agglomeration method focuses on minimizing the Sum of Squares involving distances between hypothetical cluster pairs resulting in compact clusters. Under the single linkage agglomeration method, the distance of the closest objects of the clusters determines the distance between cluster pairs. This method tends to string reduced matrix rows together to form clusters, and the resulting clusters tend to represent long chains. The complete linkage method determines distances between a cluster pair as the greatest distance between any two reduced matrix rows of a cluster pair. The complete linkage method usually performs well in cases when the objects of the clusters actually form naturally distinct groups. If the clusters tend to be somewhat elongated or of a chain type, then this method is typically not as appropriate.

Two forms of the centroid agglomeration method included the unweighted pair-group centroid method and the weighted pair-group centroid method a.k.a. the median method. The unweighted pair-group centroid method uses a centroid of a cluster that is the average point in multidimensional space defined by the dimensions of the objects in the cluster. As stated, in the case the present invention, these dimensions are the particular measurement types associated with each reduced matrix row. The centroid can be thought of as the center of gravity for the respective cluster. In this method, the distance between two clusters is determined as the difference between centroids. This method is also referred to as unweighted pair-group method using the centroid average.

The weighted pair-group centroid or median method differs from the unweighted pair-group centroid method in that weighting is introduced into the computations to take into consideration differences in cluster size which are directly related to the number of objects contained therein. When it is likely that there may be considerable differences in cluster sizes, this method is typically preferred instead of the unweighted version. Other agglomeration hierarchical clustering methods can also be used such as those having combined characteristics of the mentioned methods or other methods.

Once hierarchical clustering is performed, the procedure 200 assigns a hierarchical cluster to each test subject row of the test matrix under step 218. As a result of these assignments, the test matrix contains an additional column wherein each test subject row contains an additional entry indicating its hierarchical cluster assignment. In the depicted embodiment, a Perl script is used to perform these manipulations on ASCII data files containing the test matrices.

With clustering accomplished, the nonhierarchical and hierarchical clustering assignments added to each test subject row of the test matrix forms an expanded test matrix. This expanded test matrix is then imported to a spreadsheet or a database depending on which branch of decision step 220 of FIG. 3 is taken. The decision step 220 branches to step 222 for a spreadsheet and step 234 for a database. Alternatively, nonhierarchical and hierarchical clustering assignments are given to each test subject row of the reduced test matrix to form an expanded reduced test matrix. Although the descriptions below specifically refer to the expanded test matrix, alternative embodiments instead use the expanded reduced test matrix. Other combinations of the test matrix with the nonhierarchical and hierarchical clustering assignments are used in further alternative embodiments.

In step 222 of FIG. 3, the expanded test matrix is imported into a spreadsheet program such as Microsoft Excel. The test matrix rows of the test matrix are then sorted based on their hierarchical assignment values in step 224. In step 226, color is assigned to each measurement value of each test matrix row based upon what range each value falls in. The above embodiment uses a Visual Basic macro for color-coding, as explained below.

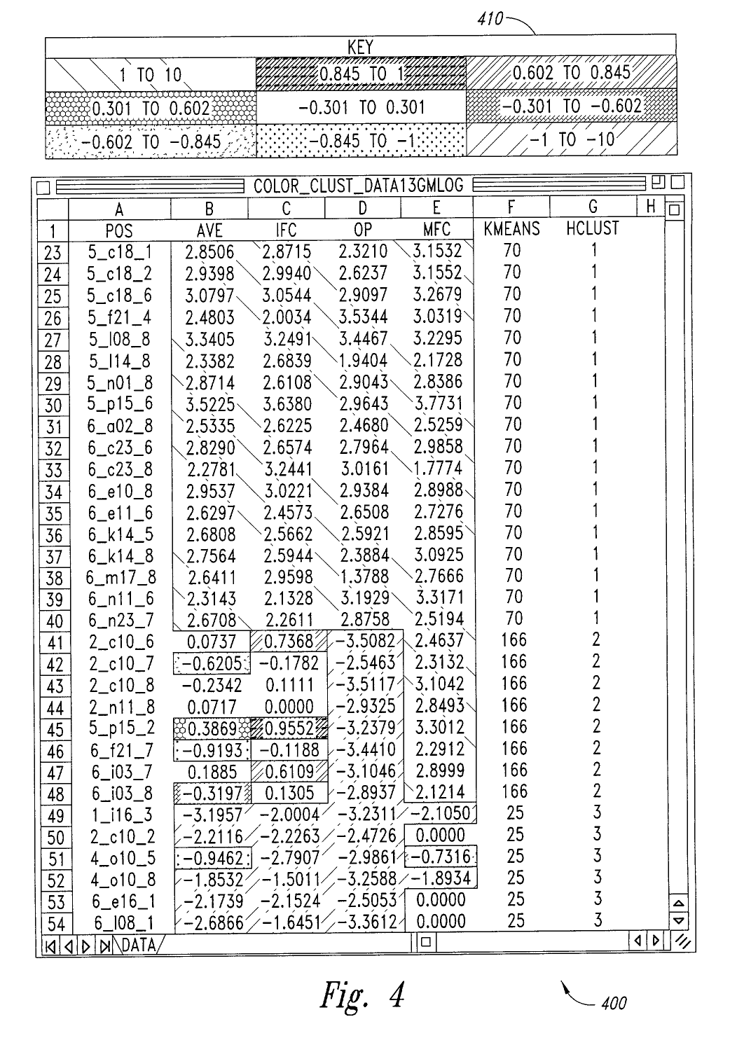

FIG. 4 illustrates a portion of an expanded test matrix as displayed in a user interface contained in a spreadsheet 400 after the individual values of the expanded test matrix have been color coded. In FIGS. 4, 6, 8, and 9 various colors are represented symbolically rather than being displayed. In the depicted embodiment, a symbolic approach is used. In alternative embodiments, actual colors are displayed to indicate the range that each measurement value falls in. For instance, referring to key 410, the ranges “1 to 10,” “0.845 to 1,” “0.602 to 0.845,” “0.301 to 0.602,” “−0.301 to 0.301,” −0.301 to −0.602,” “−0.602 to −0.845,” “−0.845 to −1,” and “−1 to −10” can be represented by the colors red, dark orange, light orange, yellow, white, light blue, dark blue, indigo, violet, respectively. Other embodiments use other color mappings between numerical ranges and colors.

Column A of the spreadsheet 400 identifies test subjects of the expanded test matrix, which in this case are actual positions in a gene array corresponding to various genes. Columns B-E contain measurement values for various types of measurements related to different brain regions conducted on the test subjects. Column B is an average of the measurement values found in columns C-E. Columns C-E are gene activity measurements of the inferior frontal cortex (IFC), the occipital pole (OP), and medial frontal cortex (MFC) brain regions, respectively. The measurements are expressed in columns C-E specifically by the following logarithm for a given gene:

$$log ( \frac{average signal in Alzheimer ' s samples}{average signal in control samples} ) .$$

Column F contains identifying numbers of the nonhierarchical cluster assignment for each test subject where the K-means nonhierarchical clustering method was used to generate the nonhierarchical cluster assignments. Alternative embodiments use other identifying labels to indicate the nonhierarchical cluster assignment for each test subject. Column G contains identifying numbers of the hierarchical cluster assignment for each test subject where HClust was used to generate the hierarchical cluster assignments. Alternative embodiments use other labels to indicate the hierarchical cluster assignment for each test subject.

The expanded test matrix rows of the spreadsheet 400 are hierarchically ordered according to their nonhierarchical cluster assignments. For instance, the nonhierarchical cluster identified by the number 70 in column F (rows 23-40) was assigned the first position as ordered by the hierarchical clustering. Similarly, the nonhierarchical cluster identified by the number 166 in column F (rows 41-48) was assigned the second position ordered by the hierarchical clustering and so on.

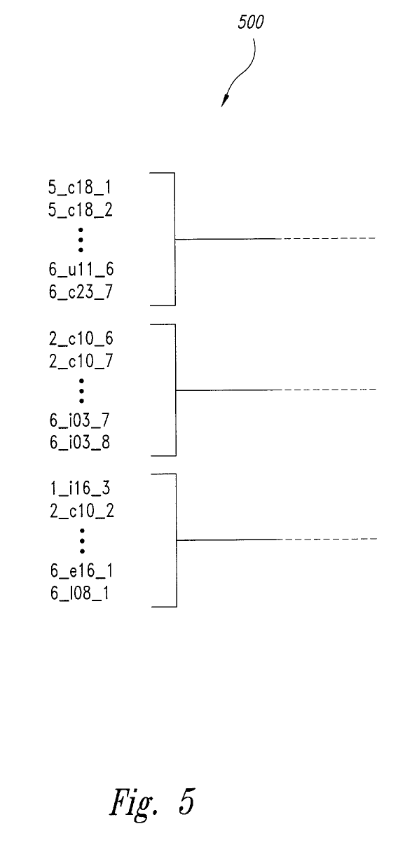

A partial dendrogram 500 shown in FIG. 5 is of the portion of the expanded test matrix rows shown in the spreadsheet 400 of FIG. 4. Both the spreadsheet 400 and the dendrogram 500 indicate how the test subjects of the test matrix are grouped in nonhierarchical clusters, however, the spreadsheet 400 provides additional information. The spreadsheet 400 reveals the basis why the test subjects are grouped in their particular nonhierarchical clusters and also the basis for the ordering of the nonhierarchical clusters established by the hierarchical clustering.

For instance, all the test subjects of the nonhierarchical cluster having cluster number 70 in column F have measurement values in the same range “1 to 10” for all measurements in columns B-E since all measurement values for these test subjects are color coded the same color. The test subjects of the nonhierarchical cluster having nonhierarchical cluster number 166 in column F have measurement values in one range “−1 to −10” for column D and measurement values in another range “1 to 10” for column E. The measurement values for the nonhierarchical cluster 166 in columns B and C are in various ranges so do not appear to be as large a factor as to why the procedure 200 grouped test subjects of nonhierarchical cluster 166 during nonhierarchical clustering. Instead, the measurement values of columns B and C support the grouping of nonhierarchical cluster 166 since, as shown by the spreadsheet 400, the measurement values in column D all fall in the range of “−1 to ‘−10” and the measurement values of column E all fall in the range of “1 to 10.” As shown by color-coded spreadsheet 400, the test subjects of nonhierarchical cluster 25 have measurement values in the same range “−1 to −10” for columns B-D which is the apparent basis for the nonhierarchical clustering of these test subjects into the nonhierarchical cluster 25.

The test subjects of nonhierarchical cluster 70 were ordered adjacent to the test subjects of nonhierarchical cluster 166 during hierarchical clustering. The test subjects of nonhierarchical cluster 70 and the test subjects of nonhierarchical cluster 166 both have measurement values in the same range “1 to 10” for column E which is the apparent reason why the procedure 200 ordered the nonhierarchical clusters 70 and 166 adjacent to one another during hierarchical clustering. The test subjects of the nonhierarchical cluster 25 are adjacent to the test subjects of the nonhierarchical cluster 166 since all the measurement values for test column D are in the same range “−1 to −10.” As these examples show, the color coding of the spreadsheet 400 effectively highlights the differences and similarities between the test subjects of an individual nonhierarchical cluster and also the differences and similarities between nonhierarchical clusters.

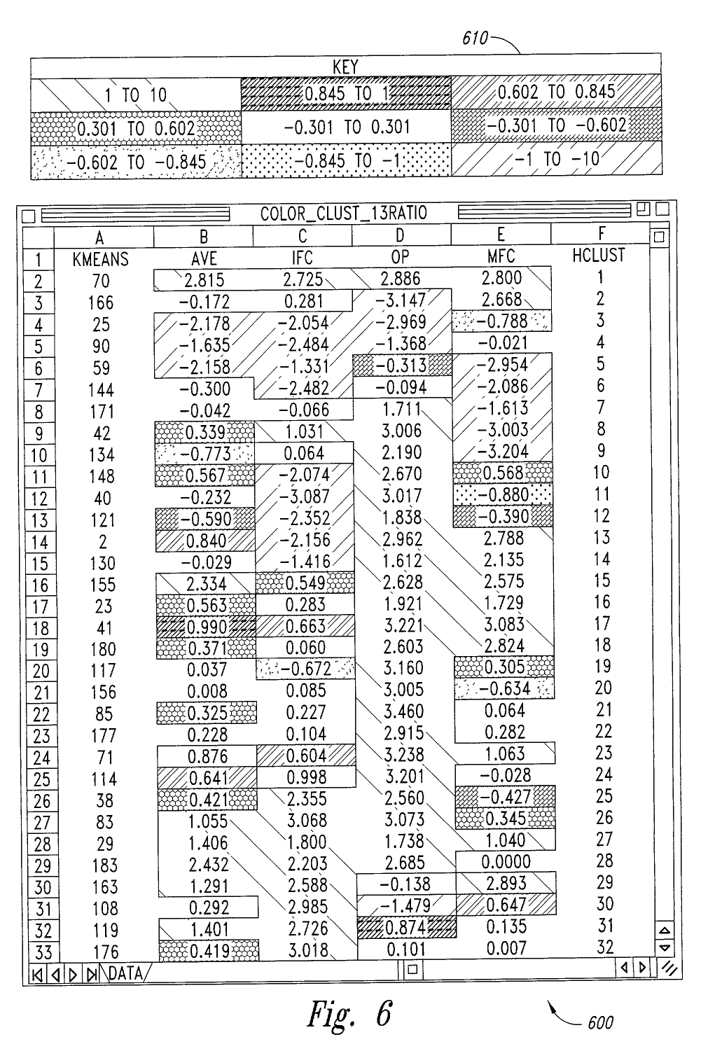

FIG. 6 shows a portion of a spreadsheet 600 of an expanded reduced test matrix that corresponds with the expanded test matrix of spreadsheet 400. The expanded reduced test matrix of spreadsheet 600 gives a focused view of how the procedure 200 orders the nonhierarchical clusters of the expanded test matrix of spreadsheet 400 under the hierarchical clustering, such as how HClust orders the K means data. In spreadsheet 600, column A contains the nonhierarchical cluster assignment numbers. Columns B-E of spreadsheet 600 contain mean values for each nonhierarchical cluster of the measurement values found in columns B-E of spreadsheet 400. For example, in spreadsheet 400 for nonhierarchical cluster 70, all the measurement values in columns B-E are in the same range of values greater than 10. In spreadsheet 600, each mean value of the expanded reduced test matrix row for nonhierarchical cluster 70 corresponds to the mean value of a column of the nonhierarchical cluster 70.

For instance, the K-means cluster 166 has eight test sample rows as shown in FIG. 4 and one corresponding reduced test sample row as shown in FIG. 6. The reduced test sample value for column B of the K-means cluster 166 shown in FIG. 6 is −0.172, which is the mean value of the eight column B values of the eight test samples for the K-means cluster 166 shown in FIG. 4.

Column F of spreadsheet 600 identifies the hierarchical ordering of the nonhierarchical cluster rows of the expanded reduced test matrix. As indicated by column F, the procedure 200 orders the rows of the spreadsheet 600 under the hierarchical clustering. The symbolic coding of the depicted spreadsheet 600 shows the basis for the hierarchical ordering of the nonhierarchical clusters. Alternative embodiments use actual color coding of portions of the spreadsheet to indicate value ranges. For instance, in the case of spreadsheet 600, many of the mean values of individual columns B-E are less than 0 for the first 15 nonhierarchical clusters. Also, for the nonhierarchical clusters in hierarchical cluster order 7 through 28, the mean values of column D all fall in the range greater than 10. The symbolic coding of the spreadsheet 600 makes these and other aspects of the mean values for the various hierarchical clusters readily apparent.

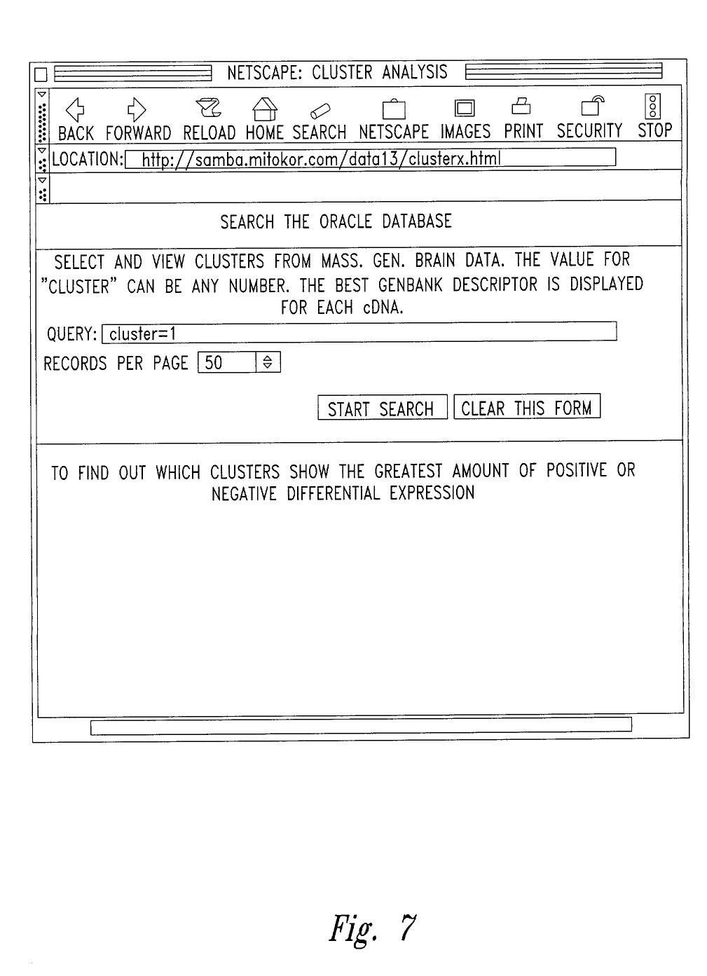

In step 232 of FIG. 3, the procedure 200 imports the expanded test matrix into a structured query language (SQL) database program such as an Oracle database. Under the above embodiments, the procedure 200 imports the hierarchical and nonhierarchical assignment data into a database containing raw gene array data. In step 234, the procedure 200 runs database queries by cluster assignments and ranking. An example of an input screen using a Web browser such as Netscape is shown in FIG. 7. Alternative embodiments include queries available under SQL or other types of query languages.

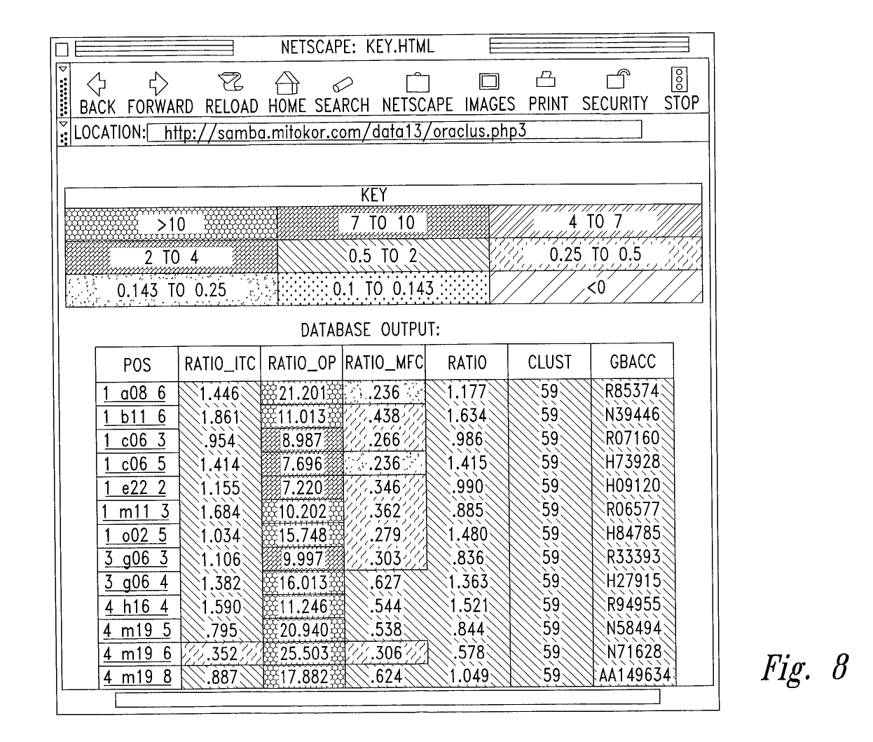

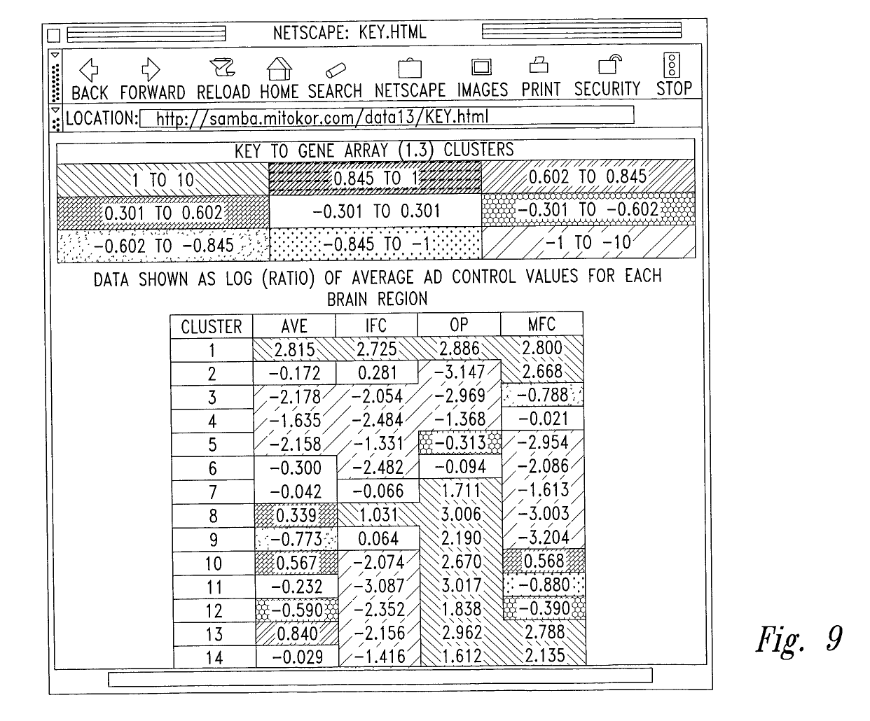

Results of the queries are displayed, for instance, in a Web browser such as Netscape, where each value of the test matrix row is color-coded as shown in FIGS. 8 and 9. FIG. 8 illustrates a user interface of the depicted embodiment involving a database query where gene activity corresponds to individual gene array portions as indicated in the “POS” column. The “ratio_ITC,” “ratio_OP,” and “ratio-MFC” columns display ratios of Alzheimer's disease values to control values for the inferior temporal cortex (ITC), occipital pole (OP), and medial frontal cortex (MFC) brain regions, respectively. The “ratio” column is an overall ratio of gene activity values to control values for the ITC, OP, and MFC brain regions, collectively. The “GBACC” column is the GenBank accession number to access a public database containing DNA sequence information. FIG. 9 illustrates a user interface of the depicted embodiment involving a database query wherein the columns identified by “cluster,” “AVE,” “IFC,” “OP,” “MFC,” and “cluster” of FIG. 9 correspond to columns B-F respectively of FIG. 6 and discussed above.

The above database uses an Apache WebServer to provide an interface to the database. A PHP hypertext preprocessor running under Linux is used to perform a SQL query on the database, and subsequently, to parse, format and color-code the resulting output in the form of a web page. Additional information on the PHP hypertext preprocessor is available at the Internet website, www.php.net. Additional information on the Apache WebServer is available of the Internet website, www.apache.org.

The above description of illustrated embodiments of the invention is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. The teachings provided herein of the invention can be applied to other clustering systems, not necessarily the hierarchical cluster analysis system and method described above.

The various embodiments described above can be combined to provide further embodiments. These and other changes can be made to the invention in light of the above detailed description. In general, in the following claims, the terms used should not be construed to limit the invention to the specific embodiments disclosed in the specification and the claims, but should be construed to include all machine vision systems that operate under the claims to provide a system or method for hierarchically clustering biological data. Accordingly, the invention is not limited by the disclosure, but instead the scope of the invention is to be determined entirely by the following claims.

It is claimed:

1. A system for analyzing information based on measurements of at least one measurement type, a measurement of each measurement type performed on each of a plurality of biological test subjects, the system comprising:
an input component configured to receive a data file of a test matrix containing sets of measurement values, each set of measurement values containing a measurement of each measurement type for one of the plurality of biological test subjects;
a pre-conditioning component configured to assign each of the sets of measurement values to one of a plurality of nonhierarchical clusters, at least one of the nonhierarchical clusters having more than one set of measurement values assigned;
a reduction component configured to generate a data file of a reduced test matrix from the data file of the test matrix, the reduced test matrix containing one set of representative values associated with each nonhierarchical cluster, each set of representative values based on the sets of measurement values assigned to the nonhierarchical cluster associated with the each set of representative values; and
a hierarchical clustering component configured to order the sets of representative values into hierarchical clusters.

2. The system of claim 1 wherein the input component receives a gene expression data file.

3. The system of claim 1 wherein the input component receives a protein expression data file.

4. The system of claim 1 wherein the input component receives two or more data files selected from the list consisting of a gene expression data file, a protein expression data file and a patient profile data file.

5. The system of claim 1 wherein the pre-conditioning component includes k-means clustering for assigning each of the sets of measurement values to one of a plurality of nonhierarchical clusters.

6. The system of claim 1 wherein the test matrix has over 10,000 rows of data.

7. The system of claim 1 wherein the data file is in ASCII file format.

8. The system of claim 1 wherein the pre-conditioning component uses Euclidean distance determinations in assigning each of the sets of measurement values to one of the plurality of nonhierarchical clusters.

9. The system of claim 1 wherein the hierarchical clustering component uses HClust of an R statistical package to order the sets of representative values into hierarchical clusters.

10. The system of claim 1 wherein an average agglomeration method is used in conjunction with the hierarchical clustering component.

11. The system of claim 1 wherein the hierarchical clustering component orders the sets of measurement values according to the ordering of the sets of representative values into hierarchical clusters.

12. The system of claim 1, further comprising a display component, the display component configured to display a portion of the sets of representative values in various colors selected according to each value of a set of representative values.

13. The system of claim 1 wherein each set of the representative values are the mean values of the sets of measurement values assigned to the nonhierarchical cluster associated with the each set of representative values.

14. The system of claim 1 wherein the plurality of nonhierarchical clusters is defined before the pre-conditioning component assigns each of the sets of measurement values to one of the plurality of nonhierarchical values.

15. An analysis system for biological data, the system comprising:
a receiver configured to receive the biological data on biological subjects, the biological subjects assigned to nonhierarchical clusters; and
a clustering component configured to hierarchically cluster the nonhierarchical clusters according to values representative of the nonhierarchical clusters.

16. The analysis system of claim 15 wherein the biological subjects are gene portions and the biological data is related to gene activity.

17. The analysis system of claim 15 wherein the biological data is associated with both control and study groups.

18. The analysis system of claim 15, further comprising a display configured to display each of the representative values in a particular color according to their value.

19. A data conditioning system comprising:
an input component configured to receive biological data on biological samples, the biological data nonhierarchically ordered according to nonhierarchical clusters of the biological samples, the nonhierarchical clusters generated by the nonhierarchical clustering system; and
a conversion component configured to generate sets of representative data for input into the hierarchical clustering system, one of the sets of representative data being generated for each nonhierarchical cluster of biological samples, each set of representative data based on the received nonhierarchically ordered biological data of the respective nonhierarchical cluster of biological samples.

20. The data conditioning system of claim 19 wherein the nonhierarchical clustering system uses unsupervised clustering and the hierarchical clustering system uses an agglomeration method.

21. The data conditioning system of claim 19 wherein the input component receives biological data based upon gene arrays.

22. The data conditioning system of claim 19 wherein the conversion component generates each set of representative data based on a mean value of the received nonhierarchically ordered biological data of the respective nonhierarchical cluster of biological samples.

23. The data conditioning system of claim 19 wherein the conversion component uses Perl script language.

24. A computer-readable medium for storing computer-readable instructions, the instructions written to program a computer to perform a method, the method comprising:
receiving a data file of biological data for biological samples;
assigning biological sample data to nonhierarchical clusters;
generating representative values for each nonhierarchical cluster; and
ordering the nonhierarchical clusters of biological data according to a hierarchical clustering based on the representative values.

25. The medium of claim 24 wherein the computer-readable medium is a CD-ROM or hard drive.

26. The medium of claim 24 wherein receiving uses a computer network.

27. The medium of claim 24, further comprising viewing in color the hierarchically clustered biological data according their values.

28. The medium of claim 24 wherein receiving a data file of biological data uses gene arrays as a data source.

29. The medium of claim 24 wherein the biological samples include study and control groups.

30. The medium of claim 24 wherein the ordering involves Perl script language for aspects including data formatting.

31. The medium of claim 24 wherein the assigning biological sample data includes biological sample data from study and control groups.

32. The medium of claim 24 wherein the assigning to nonhierarchical clusters uses distances determinations based upon at least one of the following methods: Euclidean, squared Euclidean, Cosine, Pearson correlation, Chebychev, Block, and Minkowski.

33. The medium of claim 24 wherein the assigning to nonhierarchical clusters involves at least one of the following cluster formation methods: sequential threshold, parallel threshold, or optimization.

34. The medium of claim 24 wherein the assigning to nonhierarchical clusters involves a K-means method or an Isodata method.

35. The medium of claim 24 wherein the ordering the nonhierarchical clusters according to a hierarchical clustering uses distance determination of the representative values, the distance determination being at least one of the following methods: Euclidean, Squared Euclidean, City-block, Manhattan distance, Chebychev distance, Power distance, or Percent disagreement.

36. The medium of claim 24 wherein the biological samples include study and control groups.

37. A system for displaying hierarchically clustered biological data comprising:
a color monitor;
a computer system coupled to the color monitor; and
a software program configured to instruct the computer system to display on the color monitor values representative of nonhierarchical clusters of biological data in a table having hierarchical cluster order, portions of the table colored according to the representative values.

38. The system of claim 37 wherein the software program instructs the computer system to display nonhierarchically clustered values of same nonhierarchical clustering in adjacent rows of the table and to display one row of a first nonhierarchical clustering adjacent to a row of a second nonhierarchical clustering, the first and second nonhierarchical clusters being in the same hierarchical cluster.

39. The system of claim 37 wherein a portion of the software program is a database or spreadsheet program wherein the table is part of a database or spreadsheet respectively.

40. The system of claim 37 wherein a portion of the software program is a web browser.

41. A data structure stored on a computer-readable medium, the data structure having a plurality of records containing information generated from biological samples, each of the records comprising:
a section containing the information generated from the biological samples;
a section containing a number or label indicating a nonhierarchical assignment; and
a section containing a number or label indicating a hierarchical assignment.

42. The data structure of claim 41 wherein the section containing the information generated from the biological samples includes information from a gene array.

43. A method for generating information based on biological samples, the method comprising:
receiving a data file of a test matrix containing sets of measurement values, each set of measurement values containing a measurement of each measurement type for one of the plurality of biological test subjects;
assigning each of the sets of measurement values to one of a plurality of nonhierarchical clusters, at least one of the nonhierarchical clusters having more than one set of measurement values assigned; and
generating a data file of a reduced test matrix from the data file of the test matrix, the reduced test matrix containing one set of representative values associated with each nonhierarchical cluster, each set of representative values based on the sets of measurement values assigned to the nonhierarchical cluster associated with the each set of representative values; and
ordering the sets of representative values into hierarchical clusters.

44. The method of claim 43 wherein receiving a data file of a test matrix is based on receiving a gene array.

45. The method of claim 43 wherein assigning each of the sets of measurement values to one of a plurality of nonhierarchical clusters is based on k-means clustering.

46. The method of claim 43 wherein receiving a data file of a test matrix is based on receiving an ASCII formatted file.

47. The method of claim 43 wherein assigning each of the sets of measurement values to one of a plurality of nonhierarchical clusters uses Euclidean distance determinations.

48. The method of claim 43 wherein ordering the sets of representative values into hierarchical clusters uses HClust of R statistical package.

49. The method of claim 43 wherein ordering the sets of representative values into the hierarchical clusters further includes ordering the sets of measurement values according to the ordering of the sets of representative values.

50. The method of claim 43, further comprising displaying a portion of the sets of representative values in various colors selected according to each value of a set of representative values.

51. The method of claim 43 wherein generating a data file of a reduced test matrix is based on each set of the representative values being mean values of the sets of measurement values assigned to the nonhierarchical cluster associated with the each set of representative values.

52. A method of displaying biological data comprising:
receiving biological data records or representative records, each biological data record associated with a biological sample, each representative record representing at least one biological data record, at least one of the representative records representing a nonhierarchical cluster of biological data records;
assigning the biological data records or representative records to a table having fields for values of the representative records respectively, each field containing one value;
ordering each placed biological data record in the table according to the nonhierarchically ordered cluster of its associated biological sample;
arranging each placed representative record or each ordered placed biological data record in the table according to a hierarchically ordered clustering based on the placed representative record or the representative record associated with the ordered placed biological data record; and
displaying portions of the table containing values of the arranged ordered placed biological data records or arranged placed representative records, the displaying of portions of the table according to predetermined key with respect to each displayed value.

53. The method of claim 52 wherein placing biological data records or representative records uses gene array data.

54. The method of claim 52 wherein placing biological data records or representative records is done into a table of a spreadsheet or a database program.

55. The method of claim 52 wherein regarding arranging according to a hierarchically ordered clustering, the placed representative record and the representative record associated with the ordered placed biological data record are based on mean values of the biological data records as associated with the biological samples assigned to the particular nonhierarchical ordered cluster associated with the representative record.
